(19) United States
(12) Patent Application Publication
(10) Pub. No.: US 2026/0086087 A1
(43) Pub. Date: Mar. 26, 2026

(54) ELECTROCHEMICAL REGENERATION OF IMMUNOSENSORS

(71) Applicant: Roche Diagnostics Operations, Inc., Indianapolis, IN (US)

(72) Inventors: Ines Filsinger, Mannheim (DE); Oscar Gutierrez-Sanz, Mannheim (DE); Eloisa Lopez-Calle, Mannheim (DE); Bhawana Thakur, Mannheim (DE)

(73) Assignee: Roche Diagnostics Operations, Inc., Indianapolis, IN (US)

(21) Appl. No.: 18/871,347

(22) PCT Filed: Jun. 2, 2023
(86) PCT No.: PCT/EP2023/064802

§ 371 Date: Dec. 3, 2024

(30) Foreign Application Priority Data
Jun. 3, 2022 (EP) 22177183.5

(51) Int. Cl. G01N 33/543; G01N 33/68

(52) U.S. Cl. CPC G01N 33/5438; G01N 33/6887; G01N 2333/4712

(57) ABSTRACT

The present invention concerns the field of re-usable immunosensors. In particular, it relates to a method for regenerating an immunosensor comprising at least one polypeptide attached to an electroconductive surface of said immunosensor, wherein the at least one polypeptide is capable of specifically binding an analyte to be detected by the immunosensor, said method comprising the step of applying to the immunosensor at a temperature selected from the temperature range from about 35° C. to about 42° C. a positive electrical potential of about 0.3 V on said electroconductive surface of the immunosensor for a time sufficient to allow regeneration. Moreover, it also relates to a regenerated immunosensor obtainable by the method of the present invention and a system and device comprising the immunosensor as described herein, wherein said device is capable of applying a positive electrical potential of about 0.3 V on said electro conductive surface of the immunosensor for a time sufficient to allow regeneration. The present invention also contemplates, in general, the use of a temperature selected from the temperature range from about 35° C. to about 42° C. and a positive electrical potential of about 0.3 V on an electro conductive surface of an immunosensor as described in any one of claims 1 to 13 for regeneration of said immunosensor.

The present invention concerns the field of re-usable immunosensors. In particular, it relates to a method for regenerating an immunosensor comprising at least one polypeptide attached to an electroconductive surface of said immunosensor, wherein the at least one polypeptide is capable of specifically binding an analyte to be detected by the immunosensor, said method comprising the step of applying to the immunosensor at a temperature selected from the temperature range from about 35° C. to about 42° C. a positive electrical potential of about 0.3 V on said electroconductive surface of the immunosensor for a time sufficient to allow regeneration. Moreover, it also relates to a regenerated immunosensor obtainable by the method of the present invention and a system and device comprising the immunosensor as described herein, wherein said device is capable of applying a positive electrical potential of about 0.3 V on said electroconductive surface of the immunosensor for a time sufficient to allow regeneration. The present invention also contemplates, in general, the use of a temperature selected from the temperature range from about 35° C. to about 42° C. and a positive electrical potential of about 0.3 V on an electroconductive surface of an immunosensor for regeneration of said immunosensor.

Regeneration of the sensor surface is an important but difficult task in immunosensing, especially if the sensor surface has to be used for multiple subsequent measurements. Non-invasive surface regeneration of an immunosensor platform is important for reducing costs and for being able to perform multiple measurements without significant loss of immunosensor sensitivity and/or specificity. Immunosensors that are suitable for multiple measurements would further offer the possibility to be adapted for continuous measurements and could even be adapted for being implantable and to be used for in vivo measurements.

Many currently followed regeneration approaches involve the application of harsh conditions to break the interactions between the analyte and its binding partner in the immunocomplex. These include the application of extreme pH, temperature, and chaotropic agents for dissociating the immunocomplexes underlying the immunosensing. In most of these approaches, an additional regeneration and/or washing solution is required to break the interactions between the analyte and its binding partner and/or to remove the released analyte from the sensor surface. This makes these approaches inconsistent with potential in vivo applications. Often, these approaches suffer from significant losses of sensor specificity and/or of affinity for the analyte and hence affect overall sensor function. Other currently known approaches utilize strong electrical fields for dissociating the immunocomplex that are associated with the drawbacks of unwanted oxygen reduction and electrochemical desorption, electrolysis of water on the electrode and unspecific adsorption of chemical components. Also these approaches are therefore inconsistent with potential in vivo applications.

U.S. Pat. No. 7,118,922 B1 discloses a method for regenerating a flexural plate-wave sensor (FPW) immunosensor. The method comprises a step of applying at least one electrical pulse to a bound antibody-antigen complex of an immunosensor. The electrical pulse may be applied by an electrophoretic manipulator comprising an electrical pulse applicator.

A number of studies report dissociating antibodies bound to surface-immobilized antigens for regenerating immunosensor surfaces. However, the dissociation of antigens from surface-bound antibodies by applying defined voltages has never been reported.

Asanov et al. report using electric fields to enhance the dissociation of antibody bound to a surface-immobilized antigen for regenerating a immunosensor surface being part of a total internal reflection fluorescence (TIRF) flow cell (Asanov et al., 1998, Anal. Chem. 70, 1156-1163). Polarization changes sweeping from −0.7 V to +1.1 V were applied to achieve dissociation of the antibody for sensor regeneration. Different modes of sweeping were applied, i.e. “square wave” polarization and “saw tooth” polarization.

Electrical control of antibody/antigen binding is also reported by Brod et al., 2008 (Sensors and Actuators B, 128, 560-565). The authors immobilize the antigen on the working electrode while the antibody is in solution. The antibody-antigen binding is monitored by electrochemical surface plasmon resonance measurements. After binding, application of a negative potential (−0.5 V versus Ag/AgCl reference electrode) causes rapid dissociation of the antibody molecules from the antigens. When the potential is removed, the antibodies rebind the antigens.

To ensure that immunosensors could be used repeatedly, Khoo and co-authors apply a negative potential to the working electrode to remove epitope/antigen bound antibody (Khoo, et al., 2016, Sensors and Actuators B, 224, 683-691). To regenerate the sensor surface of the immunosensor, electrode polarization was set at −0.8 V (versus Ag/AgCl, 3 M KCI) for a 10 min time interval. This way, the authors were able to dissociate the antibodies from the surface-immobilized epitopes/antigens and achieve regeneration of the immunosensor.

Up to now, negative potentials or potential sweeps covering a wide voltage range have been successfully used for dissociating immunocomplexes in which the antigen was immobilized on the electrode surface. However, the dissociation of antigens from surface-bound antibodies would offer a much wider scope of application as many commercially available immunosensor systems are based on surface-immobilized antibodies as specific binding partners for the analyte (i.e. the antigen).

Moreover, applying a negative potential as in the currently known approaches, in particular of or below −0.6 V, may have the disadvantage that biological components associated with the electrode or sensor surface may promote unwanted oxygen reduction and electrochemical desorption. On the other side, applying a high positive potential above +1 V or higher is also disadvantageous as it favors electrolysis of water on the electrode and unspecific adsorption of chemical components.

It would hence be highly desirable to provide an immunosensor regeneration method that overcomes these disadvantages.

The technical problem underlying the present invention may be seen as the provision of means and methods for complying with the aforementioned needs. It is solved by the embodiments characterized in the claims and herein below. This problem is in particular addressed by a method for regenerating an immunosensor, a regenerated immunosensor obtainable by said method and a device comprising the same, with the features of the independent claims. Advantageous embodiments which might be realized in an isolated fashion or in any arbitrary combinations are listed in the dependent claims and throughout the specification.

The method by the present invention is advantageous for at least the following reasons: The approach taken by the present inventors reduces the need of applying a potential with high intensity to the immunosensor. The invention provides an improved electrochemical regeneration method by modulating the temperature at which a moderate positive potential is applied. Moreover, the regeneration method of the invention has been demonstrated in a configuration in which the polypeptide capable of specifically binding an analyte to be detected by the immunosensor is attached to an electroconductive surface as in many commercially available sensor configurations. This offers a wide range of commercial applications. The method according to the invention may even be applied with immunosensors that measure in vivo in a continuous manner in an assay for which not only the monitoring of an increase of the analyte is important but also the monitoring of a decrease in analyte concentration.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. With respect to temperature values given in ° C., the term “about” typically refers to a deviation of +/−0.5° C. to the given value.

Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like.

Further, as used in the following, the terms “preferably”, “more preferably”, “most preferably”, “particularly”, “more particularly”, “specifically”, “more specifically”, “typically”, “more typically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The definitions made above apply mutatis mutandis to the following. Additional definitions and explanations made further below also apply for all embodiments described in this specification mutatis mutandis (except where otherwise noted).

The present invention relates to a method for regenerating an immunosensor comprising at least one polypeptide attached to an electroconductive surface of said immunosensor, wherein the at least one polypeptide is capable of specifically binding an analyte to be detected by the immunosensor, said method comprising the step of applying to the immunosensor at a temperature selected from the temperature range from about 35° C. to about 42° C. a positive electrical potential of about 0.3 V on said electroconductive surface of the immunosensor for a time sufficient to allow regeneration.

The term “sensor” as used herein is a broad term and is to be given its ordinary and customary meaning to a person of ordinary skill in the art and is not to be limited to a special or customized meaning. The term specifically may refer, without limitation, to an arbitrary element or device configured for detecting at least one condition or for measuring at least one measurement variable, typically an analyte as defined elsewhere herein. The sensor specifically may be or may comprise an analyte sensor for at least partial implantation into a body tissue of a user, more specifically an analyte sensor for continuous monitoring of the analyte. In particular, a sensor in line with the present invention is understood as referring to an immunosensor defined elsewhere herein.

The term “immunosensor” as used herein relates to a sensor capable of detecting an analyte by specific binding to a polypeptide attached to an electroconductive surface. Typically, the specific binding results in the formation of an immunocomplex on the electroconductive surface comprising at least the at least one polypeptide capable of specifically binding an analyte to be detected by the immunosensor and the analyte. Hence, an immunosensor may be regarded as a sensor wherein analyte detection is based on the formation of an immunocomplex. Further details concerning such sensors are specified elsewhere herein.

The term “specific binding” may typically relate to the binding of an antibody to an antigen. However, alternatively, the binding of an enzyme to its substrate, the binding of a matrix protein to its binding partner, or the binding between a receptor protein and its ligand are also included. More typically, “specific binding” is characterized by a certain binding affinity of said at least one polypeptide for the analyte. The binding affinity may typically be in the submolar range; more typically in the nanomolar range. Methods to determine the binding affinity of said at least one polypeptide for the analyte are known in the art and are typically based on ligand binding assays such as fluorescence quenching, isothermal titration calorimetry or surface plasmon resonance measurements (The Immunoassay Handbook, Fourth Edition, Elsevier 2013, ISBN 978-0-08-097037-0, chapter 2.12) The “immunocomplex” referred to herein, relates to said at least one polypeptide having bound the analyte. Hence, the immunocomplex according to the present invention typically comprises at least the following two components: (i) said at least one polypeptide which is capable of specifically binding an analyte to be detected by the immunosensor, and (ii) the analyte. More typically, the immunocomplex is immobilized on the electroconductive surface. Said immobilization may typically result from the at least one polypeptide being attached to the electroconductive surface of said immunosensor. More typically, the at least one polypeptide being attached to the electroconductive surface of the immunosensor and which is capable of specifically binding an analyte to be detected by the immunosensor is selected from the group consisting of: an antibody, a receptor protein, a ligand protein, a matrix protein, an enzyme, and a nucleic acid binding protein, and/or fragments thereof, even more typically said polypeptide is an antibody or fragment thereof. Hence, the formation of the immunocomplex according to the present invention typically takes place at the electroconductive surface of the immunosensor

The electroconductive surface may in particular relate to a surface of the working electrode of the sensor. More particularly, said sensor surface is exposable to the analyte or to a sample or body fluid suspected to contain the analyte.

The term “electroconductive surface” as used herein refers to a surface comprising or consisting of a material capable of conducting or transferring an electric current. Suitable materials to be used as an electroconductive surface may be or may comprise at least one metal, such as one or more of gold, copper, silver, palladium or platinum, particularly gold. Additionally or alternatively, the electroconductive surface may be or may comprise at least one electrically conductive compound, such as at least one electrically conductive organic or inorganic compound. As an example, compounds such as Ag/AgCl and indium tin oxide (ITO) may be mentioned. Additionally or alternatively, the at least one electroconductive surface may be or may comprise at least one nonmetallic electrically conductive material, e.g. graphite, carbon paste, graphene or quartz. The electroconductive surface may be or may comprise a layer of suitable electroconductive material. Typically, the electroconductive surface comprises gold or is a gold layer, a gold coating or a coating comprising gold and other compounds, like nanoparticles, or ITO. The terms “gold layer” and “gold coating” are used interchangeably herein. Typically, the terms refer to an at least one layer of gold atoms deposited onto a solid surface such as an electrode surface. Methods for atomic deposition of gold are well-known in the art and include chemical vapor deposition technologies such as atomic layer deposition. The gold layer or coating is understood to at least partially cover the electroconductive surface. A typical electroconductive surface in line with the present invention is a gold-coated quartz crystal such as commercially available from Biolin Scientific AB (Stockholm, Sweden) or the surface of a gold electrode. Further typical electroconductive surfaces in line with the present invention are graphene surfaces, or the surfaces of a carbon electrode or of an indium tin oxide (ITO) electrode.

Typically, the immunosensor to be regenerated according to the invention comprises at least one working electrode and at least one polypeptide capable of binding to the analyte. More typically, the immunosensor further comprises a counter electrode, alternatively it may further comprise a counter electrode and a reference electrode. Ever more typically, the immunosensor to be regenerated according to the invention comprises at least one working electrode and at least one polypeptide capable of binding to the analyte and a counter electrode. An immunosensor comprising two electrodes, a working electrode and a counter electrode, may also be referred to as two-electrode system elsewhere herein.

The term “regenerating an immunosensor” as used herein refers to dissociating the analyte bound to the immunosensor surface from said surface. In particular, said term refers to dissociating the analyte from the at least one polypeptide attached to the electroconductive surface of the sensor. Hence, said term may be understood as recovering the binding capacity of the electroconductive surface of the immunosensor. More particularly, the term refers to disrupting the specific binding of the analyte to the at least one polypeptide. In other words, the term “regenerating an immunosensor” in line with the present invention may be understood as referring to the unbinding or detaching of the analyte from said at least one polypeptide which is attached to the electroconductive surface of the sensor. More particularly, the term “regenerating” refers to dissociating the immunocomplex comprising the polypeptide capable of specifically binding the analyte to be detected by the immunosensor and the analyte bound to this polypeptide. The dissociation results in recovering of the binding capacity of the immunosensor, particularly the binding capacity of the least one polypeptide attached to the electroconductive surface of the sensor and being capable of specifically binding an analyte to be detected by the immunosensor. Typically, the regeneration according to the method of the present invention recovers the binding capacity of the immunosensor up to at least 60%, at least 70% or at least 75% compared to a control immunosensor. Said “recovered binding capacity” is also referred to as “regenerated binding capacity” herein. The relative amount of sensor recovery [%] may be calculated as the ratio of the amount of analyte (e.g. of TSH) bound to the immunosensor after regeneration to the amount of the analyte bound by a fresh immunosensor multiplied by 100%.

The term “control immunosensor” typically refers to an immunosensor that has not been previously in contact with a sample and/or the analyte. A “control immunosensor” is also referred to as “fresh immunosensor” herein. The term “control immunosensor” may also refer to an immunosensor that has been previously in contact with a sample and/or the analyte, but has been treated thereafter by chemical and/or physical methods in a way that essentially all bound analyte molecules have been removed from the least one polypeptide attached to the electroconductive surface of the immunosensor.

The binding capacity can be determined by various methods known to the skilled artisan. Suitable methods include: microbalance detection, particularly quartz crystal microbalance (QCM) detection, surface plasmon resonance measurements, electrochemical impedance spectroscopy (EIS) measurements, total internal reflection fluorescence measurements. Typically, a microbalance detection method, particularly a quartz crystal microbalance (QCM) detection method, may be performed for determining the binding capacity of an immunosensor. Briefly, an immunosensor may be brought into contact with a sample containing a known amount of the analyte; typically an amount sufficient to achieve sensor saturation. In particular, sensor saturation may be achieved with samples containing a concentration of the analyte exceeding 10 times the K_{D} of the polypeptide capable of specifically binding an analyte to be detected by the immunosensor. Thereby, it is thought that 100% of the binding sites on the sensor surface will bind an analyte. This means that essentially all of the available polypeptides which are capable of specifically binding an analyte to be detected by the immunosensor and which are attached to the electroconductive surface will bind to an analyte. The weight of bound analyte is typically determined by microbalance detection and reflects the binding capacity of the immunosensor. More typically, the total amount of analyte is specified as mass bound per area (ng/cm²); said value specifically reflects the mass of analyte bound to the electroconductive surface of the immunosensor. Said value determined with a fresh immunosensor under said sensor saturation conditions shall be set to 100% when comparing to the binding capacity of regenerated immunosensors. A suitable instrument for microbalance measurements is for example the commercially available Q-sense E4 from Biolin Scientific AB (Stockholm, Sweden) coupled with an electrochemistry module. The mass deposited on the surface may be extracted using a suitable software tool such as Dfind software (Biolin Scientific AB (Stockholm, Sweden).

The terms “protein” or “polypeptide” are used interchangeably and refer to molecules consisting of a chain of amino acids, without reference to a specific mode of action, size, 3-dimensional structure or origin.

The phrase “capable of specifically binding an analyte to be detected by the immunosensor” generally refers to the binding affinity of the at least one polypeptide to the analyte to be detected by the immunosensor.

As commonly known, the affinity of a polypeptide, as defined elsewhere herein, to its binding partner, such as an antigen or an analyte, is inversely related to the dissociation constant (K_{D}). A high affinity of the at least one polypeptide for the analyte to be detected by the immunosensor is hence characterized by a low K_{D}. Typically, a K_{D} of below 10 μM represents a suitable affinity for the binding of the polypeptide to an analyte to be detected by the immunosensor in line with the present invention. More typically, a K_{D} in the range of in between 10⁻¹⁵ M to 10⁻⁵ M may represent a suitable affinity in line with the present invention.

The “at least one polypeptide which is capable of specifically binding an analyte to be detected by the immunosensor” shall be attached to the electroconductive surface. Typically, attaching the polypeptide to the electroconductive surface results in the immobilization of said polypeptide on said surface.

Hence, in accordance with the present invention the “at least one polypeptide which is capable of specifically binding an analyte to be detected by the immunosensor” may be understood to be immobilized or configured to become immobilized on the electroconductive surface of the sensor. More typically, the polypeptide is attached by a covalent chemical bond to the electroconductive surface. Even more typically, said covalent bonding may be achieved by covalent linkage to the electroconductive surface through chemical linkers such as widely used EDC/NHS chemistry involving N-ethyl-N′-(3-(dimethylamino) propyl) carbodiimide (EDC) and N-hydroxysuccinimide (NHS). Further ways of attaching the polypeptide by a covalent chemical bond to the electroconductive surface include thiol coupling or conjugations. The skilled artisan is well aware of the procedures necessary for establishing said attachment by said chemical linker molecules. The kind of chemical linker molecules used may be adapted based on the polypeptide to be attached to the electroconductive surface and to the electroconductive material of the surface.

Typically, said at least one polypeptide has bound an analyte prior to carrying out the method according to the invention. In other words, typically, prior to carrying out the method according to the invention the analyte to be detected by the immunosensor is bound to the polypeptide capable of specifically binding said analyte. More typically, the immunosensor has been in contact with a sample or a body fluid suspected to contain the analyte prior to carrying out the method according to the invention. Even more typically, the at least one polypeptide of the immunosensor has specifically bound an analyte contained in a sample or in a body fluid. Still even more typically, said binding has resulted in the detection of the analyte by said immunosensor.

The term “sample” is understood by the skilled person and typically relates to any subportion of a body fluid. Samples can be obtained by well known techniques including, e.g., venous or arterial puncture, epidermal puncture, and the like. Samples may be processed by any technical means prior to contacting with the immunosensor including, but not limited to: heating, diluting, centrifuging, filtering, pH adjusting, and the like. Alternatively, in particular in case of in vivo measurements, the immunosensor may be in direct contact with the body fluid suspected to contain the analyte. More particularly, in case of implantable or implanted immunosensors, the immunosensor may be in direct contact with the body fluid suspected to contain the analyte.

As used herein, the term “body fluid” relates to all body fluids of a subject known to comprise or suspected to comprise the analyte of the present invention, including interstitial fluid, blood, plasma, lacrimal fluid, urine, lymph, cerebrospinal fluid, bile, stool, sweat, and saliva. Generally, an arbitrary type of body fluid may be used. Preferably, the body fluid is a body fluid which is present in a body tissue of a subject, such as in the interstitial tissue. Thus, as an example, the body fluid may be selected from the group consisting of blood and interstitial fluid, typically interstitial fluid. However, additionally or alternatively, one or more other types of body fluids may be used. The body fluid generally may be contained in a body tissue.

Also typically, said polypeptide which is capable of specifically binding an analyte to be detected by the immunosensor is selected from the group consisting of: an antibody, a receptor protein, a ligand protein, a matrix protein, an enzyme, and a nucleic acid binding protein, and/or fragments thereof. More typically, said polypeptide comprises an antibody or fragment thereof, even more particularly an IgG or a Fab fragment. Still even more typically, said polypeptide is a Fab fragment specifically recognizing a polypeptide analyte. Particularly, said analyte may be selected from TSH and TnT, and suitable examples of polypeptides which are capable of specifically binding an analyte to be detected by the immunosensor include anti-TSH F(ab′)2 antibody fragment and anti-TnT fab antibody fragment, available e.g. from Roche Diagnostics GmbH, Penzberg, Germany.

According to the method of the present invention, a positive electrical potential shall be applied to the immunosensor and, in particular, the at least one polypeptide attached to the electroconductive surface. The electrical potential shall be in the range of about +0.1 to about +0.5 V, typically in the range of about +0.2 to +0.4 V. More typically, the electrical potential shall be about +0.3 V. In particular, a two-electrode system may be used as defined elsewhere herein. Said potential or a potential within said range is advantageous as it may contribute to the dissociation of the immunocomplex without harming or damaging the polypeptide and/or altering its binding capacity and/or binding specificity. It is thought that applying said potential, the secondary structure and/or the net charge of said polypeptide may be amended. This may interfere with the electrostatic interactions involved in the binding of said at least one polypeptide to the analyte and is expected to promote unbinding and dissociation of the immunocomplex. Further, it was found by the inventors that applying a negative potential lead to irreversible changes of the senor surface.

The moderate positive potential applied in the method of the invention avoids or reduces undesired side effects including unwanted oxygen reduction, electrochemical desorption, electrolysis of water on the electrode and unspecific adsorption of chemical components. Applying a moderate positive potential within the herein described range is hence advantageous.

The potential shall be applied by using the electroconductive surface as one electrode and at least one additional electrode, particularly as a counter-electrode. Alternatively, the at least one polypeptide attached to the electroconductive surface may be placed into an electrical field between two electrodes applying the electrical potential. The skilled person is well aware of how an electrical potential can be applied to the at least one polypeptide attached to the electroconductive surface of the immunosensor according to the present invention. Typically, the electrical potential is applied and may be controlled by using an analog output, such as for example a voltage source, a current source, a voltage meter, a current meter, an impedance meter, an impedance spectrometer, a frequency analyzer, a potentiostat, a frequency generator. or a battery.

The electrical potential is applied for a time sufficient to allow immunosensor regeneration. Typically, the electrical potential is applied for a time being selected from the range of about 5 min to about 30 min; more typically for a time being selected from the range of about 7 min to about 20 min, even more typically for a time being selected from the range of about 8 min to about 12 min. Depending on the degree of regeneration which is envisaged, the time for which the electrical potential is applied may be reduced or increased. More typically, said time is about 10 min.

Moreover, the electrical potential shall be applied at a temperature selected from the temperature range from about 35° C. to about 42° C. The temperature can be applied and/or controlled actively, i.e. by applying heat to the immunosensor until the desired temperature within the temperature ranges is reached and maintained. Alternatively, the immunosensor may be placed into an environment having the desired temperature such that the temperature of the immunosensor passively adjusts to the environmental temperature. Typically, this is the case if an immunosensor is used in an in vivo application within a subject having a body temperature within in the aforementioned temperature range. More typically, the temperature envisaged is in the range of 36° C. to 40° C., even more typically the temperature envisaged is about 37° C. Applying said temperature is advantageous as it contributes to dissociating the analyte from the at least one polypeptide which is capable of specifically binding an analyte to be detected by the immunosensor. The inventors hypothesize that the temperature may result in or facilitate structural and/or conformational changes of said polypeptide and/or of said analyte that may loosen the interactions between said polypeptide and the analyte in the immunocomplex. The effect of the temperature on the dissociation of the immunocomplex is particularly pronounced in combination with the moderate potential applied; in other words applying said temperature typically allows for efficient dissociation of the immunocomplex already at a moderate potential. This is advantageous as the use of harsh conditions that are potentially harmful to the components of the immunocomplex, namely said at least one polypeptide which is capable of specifically binding an analyte to be detected by the immunosensor, and the analyte, may be avoided. This offers the possibility of regenerating an immunosensor multiple times using relatively mild conditions while preserving its sensitivity and specificity.

If the immunosensor is used ex-vivo, the immunosensor may also typically be treated with a washing solution in order to remove detached analyte from the immunosensor. Thus, typically, the immunosensor is treated with a pH-neutral buffer solution, more typically at least the electroconductive surface of the immunosensor is treated with a pH-neutral buffer solution.

Even more typically, said buffer solution is applied for removing detached analyte. Most typically, said buffer solution is a 50 mM 4-Morpholineethanesulfonic acid (MES) solution, adjusted to pH 7.0. Specifically, the buffer solution is applied for a period of time sufficient to remove detached analyte, for example for a period of time in the range of about 1 min to about 30 min. More specifically, the buffer solution is applied to the electroconductive surface in a microfluidic system such as a flow cell. In the case of using a microfluidic system, the surface of the immunosensor may be rinsed with the buffer solution in a continuous manner. Typically, the buffer solution may be contained in a reservoir in fluid connection with the electroconductive surface of the sensor. More typically, the senor or at least the electroconductive surface of the sensor is located in a flow cell being in fluid connection with said reservoir. Such microfluidic system are advantageous as they may support particularly efficient removal of detached analyte.

It will be understood that regeneration in an in vivo used immunosensor does not require such washing using a pH-neutral buffer solution.

Particularly, in the case of in vivo use, the immunosensor according to the present invention may be implantable, fully or a partially, and may, thus, be adapted for performing the detection of the analyte in the body fluid in a subcutaneous tissue, in particular, in an interstitial fluid. Other parts or components may remain outside of the body tissue. For example, as used herein, the terms “implantable” or “subcutaneous” refer to be fully or at least partly arranged within the body tissue of the subject. For this purpose, the immunosensor may comprise an insertable portion, wherein the term “insertable portion” may generally refer to a part or component of an element configured to be insertable into an arbitrary body tissue. Typically, the insertable portion may fully or partially comprise a bio-compatible surface layer or coating, which may have as little detrimental effects on the user or the body tissue as possible, at least during typical durations of use. For this purpose, the insertable portion may be fully or partially covered with at least one biocompatibility membrane layer, such as at least one polymer membrane, for example a gel membrane which, on one hand, may be permeable for the body fluid or at least for the analyte to be detected by the immunosensor, and may on the other hand be impermeable for compounds comprised in the immunosensor, in particular in the working electrode, thus preventing a migration thereof into the body tissue. Suitable biocompatible materials for use as the biocompatibility membrane layers are known in the art and may include one or more of the following materials: methacrylate based polymers and copolymers, acrylamide-methacrylate based copolymers, biodegradable polysaccharides such as hyaluronic acid (HA), agarose, dextran and chitosan, and polyvinylpyrrolidone.

In the method according to the present invention said at least one polypeptide which is capable of specifically binding an analyte to be detected by the immunosensor specifically recognizes an analyte. Specifically, said polypeptide is an antibody or fragment thereof. The terms “specific” recognizing” and “specific binding” are used interchangeably and described elsewhere herein in more detail.

The term “analyte” as used herein is a broad term and is to be given its ordinary and customary meaning to a person of ordinary skill in the art and is not to be limited to a special or customized meaning. The term specifically may refer, to an arbitrary element, component or compound which may be present in a body fluid and the concentration of which may be of interest for e.g. a subject or a healthcare professional. Specifically, the analyte may be or may comprise an arbitrary chemical substance or chemical compound which may take part in the metabolism of a subject or patient. As an example, the at least one analyte may be selected from the group consisting of: polypeptides, small molecules, carbohydrates, lipids. More specifically, the analyte is a protein or polypeptide. Additionally or alternatively, however, other types of analytes may be used and/or any combination of analytes may be determined.

Typical polypeptide analytes include cardiac troponin molecules or derivatives thereof, particularly troponin T or troponin I and derivatives thereof, Brain Natriuretic Peptide (BNP) molecules or derivatives thereof, particularly BNP or nt-proBNP and derivatives thereof, D-dimer molecules; peptide hormones such as thyreotropin (TSH), human choriongonadotropin (hCG), erythropoietin (EPO), insulin, somatotropin (human growth hormone, hGH), adrenocorticotropin (ACTH).

More specifically, in the method according to the present invention said at least one polypeptide may be specifically recognizing a cardiac troponin as the analyte and said analyte may be said cardiac troponin. Typically, said cardiac troponin is troponin T. In this case, the at least one polypeptide which is capable of specifically binding an analyte to be detected by the immunosensor is an anti-troponin antibody or fragment thereof, for example an anti-TnT fab antibody fragment.

Alternatively, in the method according to the present invention said at least one polypeptide may be specifically recognizing TSH as the analyte and said analyte may be said TSH. In this case, the at least one polypeptide which is capable of specifically binding an analyte to be detected by the immunosensor is an anti-TSH antibody or fragment thereof, for example an anti-TSH F(ab′)2 antibody fragment.

The method according to the invention may comprise, at least once, a further step of measuring using the regenerated immunosensor and a further step of applying to the immunosensor at a temperature selected from the temperature range from about 35° C. to about 42° C. a positive electrical potential of about 0.3 V on said electroconductive surface of the immunosensor for a time sufficient to allow regeneration after measuring. In other words, the method may comprise a further step of detecting the analyte using the regenerated immunosensor and a further step of regenerating the immunosensor by applying the described temperature and potential. These further steps may be regarded as a second regeneration cycle. The present invention hence provides means for more than one cycle of regeneration. The number of regeneration cycle is in principle not limited. Typically, the method according to the invention may offer means for regenerating an immunosensor multiple times such as two times, three times, four times, five times, up to ten times, or more.

The present invention also relates to a regenerated immunosensor obtainable by the method of the present invention. The regenerated immunosensor typically has a binding capacity of at least 60%, at least 70% or at least 75% of a control immunosensor. The person skilled in the art knows how the binding capacity of the immunosensor may be determined. Exemplified procedures for determining the binding capacity are described elsewhere herein in more detail.

Moreover, the present invention relates to a method for detecting an analyte in a sample or a body fluid using the regenerated immunosensor described herein above.

Yet, the present invention relates to a device comprising the immunosensor as described herein, wherein said device is capable of applying a positive electrical potential of about 0.3 V on said electro conductive surface of the immunosensor for a time sufficient to allow regeneration. Moreover, said device may typically be capable of applying said potential at a temperature selected from the temperature range from about 35° C. to about 42° C. More typically, said device may comprise a heat source capable of actively heating and controlling the immunosensor to said temperature. Alternatively, said device may comprise a heat source capable of heating the environment surrounding the immunosensor to passively adjust the temperature of the immunosensor to the environmental temperature. In case the immunosensor is implantable and/or is implanted, said heat source may not be necessary.

The device may further include at least one analog output such as a voltage source, a current source, a voltage meter, a current meter, an impedance meter, an impedance spectrometer, a frequency analyzer, a potentiostat, a frequency generator, or a battery. The analog output may in particular be used to apply and control the above specified positive potential.

Typically, the device comprises at least one evaluation unit comprising at least one data processing unit, such as a microcontroller.

The device typically comprises a housing and may further comprise a user interface or means for data storage and/or data transfer. The user interface may comprise a display. More typically, the device comprises one or several control elements. The device is typically part of a system adapted to perform the method for regenerating an immunosensor of the present invention.

Further, the device may comprise an analysis channel or region wherein the electroconductive surface of the immunosensor is disposed to be in contact with the sample, typically the body fluid.

The sensor may be combined with further sensing technologies including quartz crystal microbalance (QCM), surface plasmon resonance (SPR), Raman spectroscopy, Fourier-transform infrared spectroscopy (FTIR) and the like. Specifically, the sensor moreover may be combined with a microbalance unit. Particularly when used in an in vitro embodiment, the sensor may be part of a fluidic system, in particular a microfluidic system, including at least a pump, one or more valve selectors.

Particularly when used in an in vitro embodiment, and when being part of a fluidic system, said device may typically comprise a reservoir for a pH-neutral buffer solution as specified elsewhere herein. The device may further comprise at least one flow cell, typically at least one electrochemical flow cell, typically in connection with a peristaltic pump. The reservoir may be in microfluidic connection with the flow cell and the peristaltic pump.

Even further, said device may be a fully or partially implantable device. Hence, said device may further comprise a biocompatibility membrane layer as described elsewhere herein. Typically, said device is adapted for in vivo measurements of an analyte specifically recognized by the at least one polypeptide of the immunosensor. More typically, said in vivo measurements are continuous or semi-continuous measurements. A semi-continuous measurement is a serious of subsequent measurements, preferably at defined time intervals (e.g. every 0.1 to 1000 seconds). More typically, said in vivo measurements are real-time in vivo measurements. Even more typically, said analyte is present and may be detectable in the interstitial fluid of a subject and the device. Even further, the device may be implanted in a suitable tissue to be in contact with the interstitial fluid.

The present invention further relates to a system adapted to perform the method for regenerating an immunosensor. Said system comprises a device as outlined elsewhere herein and at least one electronic control element. Said electronic control element is capable of controlling at least the parameters applied in the method of the present invention. Said parameters include the positive electrical potential, the time of applying the potential, and typically in addition also the temperature. Said electronic control element typically is a data processing unit, for example a microcontroller.

The present invention also contemplates, in general, the use of a temperature selected from the temperature range from about 35° C. to about 42° C. and a positive electrical potential of about 0.3 V on an electroconductive surface of an immunosensor as described elsewhere herein for regeneration of said immunosensor. Said use is advantageous as it allows for efficient regeneration of an immunosensor.

The following are preferred embodiments envisaged by the present invention:

Embodiment 1: A method for regenerating an immunosensor comprising at least one polypeptide attached to an electroconductive surface of said immunosensor, wherein the at least one polypeptide is capable of specifically binding an analyte to be detected by the immunosensor, said method comprising the step of applying to the immunosensor at a temperature selected from the temperature range from about 35° C. to about 42° C. a positive electrical potential of about 0.3 V on said electroconductive surface of the immunosensor for a time sufficient to allow regeneration.

Embodiment 2: The method of embodiment 1, wherein said at least one polypeptide has bound said analyte prior to carrying out the method.

Embodiment 3: The method of embodiment 1 or 2, wherein said polypeptide which is capable of specifically binding said analyte to be detected by the immunosensor is selected from the group consisting of: an antibody, a receptor protein, a matrix protein, an enzyme, and a nucleic acid binding protein.

Embodiment 4: The method of any one of embodiments 1 to 3, wherein said time is selected from the range of about 8 to about 12 min.

Embodiment 5: The method of embodiment 4, wherein said time is about 10 min.

Embodiment 6: The method of any one of embodiments 1 to 5, wherein the immunosensor is treated with a pH-neutral buffer solution.

Embodiment 7: The method of embodiment 6, wherein said buffer solution is applied for removing detached antigens.

Embodiment 8: The method of embodiment 6 or 7, wherein said buffer solution is a 50 mM 4-Morpholineethanesulfonic acid (MES) solution, adjusted to pH 7.0.

Embodiment 9: The method of any one of embodiments 1 to 8, wherein the binding capacity of the immunosensor can be recovered up to at least 60%, at least 70% or at least 75% compared to a control immunosensor.

Embodiment 10: The method of any one of embodiments 1 to 9, wherein said at least one polypeptide is specifically recognizing a cardiac troponin and said analyte is said cardiac troponin.

Embodiment 11: The method of embodiment 10, wherein said cardiac troponin is troponin T.

Embodiment 12: The method of any one of embodiments 1 to 9, wherein said at least one polypeptide is specifically recognizing TSH and said analyte is TSH.

Embodiment 13: The method of any one of embodiments 1 to 11, wherein said method comprises at least once a further step of measuring using the regenerated immunosensor and a further step of applying to the immunosensor at a temperature selected from the temperature range from about 35° C. to about 42° C. a positive electrical potential of about 0.3 V on said electro conductive surface of the immunosensor for a time sufficient to allow regeneration after measuring.

Embodiment 14: A regenerated immunosensor obtainable by the method of any one of embodiments 1 to 13.

Embodiment 15: A device comprising the immunosensor according to embodiment 14, wherein said device is capable of applying a positive electrical potential of about 0.3 V on said electro conductive surface of the immunosensor for a time sufficient to allow regeneration.

Embodiment 16: The device of embodiment 15, wherein said positive electrical potential is applied at a temperature a temperature selected from the temperature range from about 35° C. to about 42° C.

Embodiment 17: The device of embodiment 15 or 16, wherein said device comprises a reservoir for a pH-neutral buffer solution as specified in any one of embodiments 6 to 8.

Embodiment 18: The device of any one of embodiments 15 or 17, wherein said device is a implantable device.

Embodiment 19: The device of any one of embodiments 15 to 18, wherein said device is adapted for continuous in vivo measurements of an analyte specifically recognized by the at least one polypeptide of the immunosensor.

Embodiment 20: The device of embodiment 19, wherein said in vivo measurements are real-time in vivo measurements.

Embodiment 21: A system adapted to perform the method according to any one of embodiments 1-13 comprising:
i) a device comprising:
   at least one immunosensor comprising at least one polypeptide attached to an electroconductive surface of said immunosensor, wherein the at least one polypeptide is capable of specifically binding an analyte to be detected by the immunosensor, and
   at least one control element capable of applying a positive electrical potential of about 0.3 V on said electroconductive surface of the immunosensor for a time sufficient to allow regeneration;
ii) at least one electronic control element capable of controlling at least the parameters applied in the method according to any one of embodiments 1-13.

Embodiment 22: The system of embodiment 21, wherein the at least one electronic control element is further capable controlling the temperature.

Embodiment 23: The system of any one of embodiments 21 or 22, wherein said polypeptide which is capable of specifically binding said analyte to be detected by the immunosensor is selected from the group consisting of: an antibody, a receptor protein, a matrix protein, an enzyme, and a nucleic acid binding protein

Embodiment 24: The system of any one of embodiments 21 to 23, wherein said immunosensor is a regenerated immunosensor obtained by the method of any one of embodiments 1 to 13.

Embodiment 25: The system of any one of embodiments 21 to 24, wherein said at least one electronic control element is a microcontroller capable of controlling at least the positive electrical potential and the time of applying the potential.

Embodiment 26: Use of a temperature selected from the temperature range from about 35° C. to about 42° C. and a positive electrical potential of about 0.3 V on an electro conductive surface of an immunosensor as described in any one of embodiments 1 to 13 for regeneration of said immunosensor.

All references cited in this specification are herewith incorporated by reference with respect to their entire disclosure content and the disclosure content specifically mentioned in this specification.

### FIGURES

FIG. 1: Electrochemical regeneration of a TSH sensor (e-QCM data). Depicted is the change in mass per area [ng/cm²] versus the time [min].

FIG. 1A: Detection of TSH with a new (fresh) sensor, non-regenerated, 57 ng of TSH was bound (this value generated with the new immunosensor is used as reference value for the calculation of regeneration efficiency)

FIG. 1B: Detection of TSH with an electrochemically regenerated TSH sensor, regeneration was performed at 40° C. and applying 0.3 V for 10 minutes, 46 ng of TSH was bound representing a regenerated binding capacity of 80.7% compared to the new sensor measurement of (A)

FIG. 1C: Detection of TSH with a conventionally TSH sensor, regeneration was performed using a glycine treatment at pH 2 for 10 minutes, 51 ng of TSH was bound representing a regenerated binding capacity of 89.5% compared to the new sensor measurement of (A)

FIG. 2: TSH sensor recovery for different regeneration conditions. The recovery rate is shown in %. The percentage of recovery refers to the regenerated binding capacity of the sensor based in comparison with a new, i.e. fresh sensor. The percentage of sensor recovery is calculated as the ratio of the amount of analyte (TSH) bound to the immunosensor after regeneration to the amount of the analyte bound by a fresh immunosensor multiplied by 100%. The amount of analyte bound by a fresh immunosensor is depicted in FIG. 1.

FIG. 3: Assessment of different regeneration approaches for a cardiac Troponin T (TnT) sensor by e-QCM measurements. Depicted is the mass per area change [ng/cm²] versus the time [min].

FIG. 3A: Detection of TnT with a new sensor, non-regenerated, 394 ng of TnT was bound;

FIG. 3B: Detection of TnT with a conventionally regenerated TnT sensor, regeneration was performed using a glycine treatment at pH 2 for 2 minutes, 51 ng of TSH was bound representing a regenerated binding capacity of 89.5% compared to a new sensor,.

FIG. 3C: Detection of TnT with an electrochemically regenerated TnT sensor; regeneration was performed at 25° C. and applying 0.3 V for 10 minutes, 62.26 ng of TnT was bound representing a regenerated binding capacity of 16.05% compared to a new sensor ;

FIG. 3D: Detection of TnT with an electrochemically regenerated TnT sensor; regeneration was performed at 37° C. and applying 0.3 V for 10 minutes, 285.94 ng of TnT was bound representing a regenerated binding capacity of 76.34% compared to a new sensor.

FIG. 4: TnT sensor recovery for different regeneration conditions. The recovery rate is shown in %. The percentage of recovery was calculated as in FIG. 2.

FIG. 5: Multiple repetitions of sensor TnT regeneration. The bar graph shows the regenerated binding capacity as % of sensor recovery. For the new (fresh) sensor the efficiency is set to 100% and sensor recovery after 1^{st}, 2^{nd} and 3^{rd} round of regeneration.

### EXAMPLES

The invention will be described by way of examples. However, the examples shall merely illustrate the invention and not be construed, whatsoever, as limiting the scope of the invention.

### Example 1: Sensor Regeneration

Chemicals: H₂O₂ (30%), NH₃ solution (25%), sodium hydroxide pellets (NaOH), 4-Morpholineethanesulfonic acid (MES), N-ethyl-N′-(3-(dimethylamino) propyl) carbodiimide (EDC), N-hydroxysuccinimide (NHS), and bovine serum albumin (BSA) were from Sigma-Aldrich, Germany. SH-PEG-COOH (0.5 kDa) was procured from Iris Biotech, Germany. Anti-TSH F(ab′)2 antibody fragments, anti-TnT fab antibody fragments, recombinant TSH antigen and recombinant TnT antigen were from Roche Diagnostics GmbH, Penzberg, Germany. Gold-coated quartz crystal was procured from Biolin Scientific AB (Stockholm, Sweden).

Instruments: Measurements were performed on Q-sense E4 from Biolin Scientific AB (Stockholm, Sweden) coupled with an electrochemistry module. The mass deposited on the surface was extracted with Dfind software (Biolin Scientific AB (Stockholm, Sweden). Ivium-n-Stat was used for electrochemical voltage pulse application.

Modification of QCM chip: The gold quartz crystal microbalance (QCM) chip was cleaned in base piranha solution (1 part 30% H₂O₂+1 part 25% NH₃+8 parts de-ionized (DI) water) for 10 min at 70° C. Followed by a thorough rinse with DI water, the chip was dried with N₂. The cleaned chip was treated with UV lamp before the modification. For modification of the chip, reagents were introduced in microfluidic flow channel (Flowrate: 80 μL/min) coupled with QCM. All the reagents were prepared in 50 mM MES buffer pH 7 unless otherwise stated. Further, the same buffer was used throughout the measurements for stabilizing the sensor and for intermediate washing steps. The buffer stabilized gold chip was treated with a solution of 0.5 kDa SH-PEG-COOH (40 μM) for 40 min (flow rate: 80 μL/min). The thiol-modified sensor chip was rinsed with buffer for 10 min. To activate the —COOH groups on the sensor, EDC (0.4 mg/mL)-NHS (0.6 mg/mL) mixture prepared in 50 mM MES buffer pH 4.7 was introduced in the flow channel for 20 min. The activated sensors were modified with either anti-TSH F(ab′)2 (170 μg/mL) or anti-TnT Fab antibody fragments (21 μg/mL) in 50 mM MES buffer pH 4.7) for 30 min. To remove the unbound antibody fragments, buffer was allowed to flow in the fluidic channel for 10 min. Bovine serum albumin (BSA; 10 mg/mL in MES pH 7) was allowed to flow for 10 min to block the remaining active sites on the gold chip.

Regeneration procedures: For glycine regeneration, 50 mM glycine pH 2 buffer was allowed to flow for 10 min (flow rate of 80 μL/ min) on the analyte-saturated sensor. This was followed by the flow of 50 mM MES buffer pH 7. Once the regenerated sensor was stabilized, i.e. once the sensor drift was around 2 Hz/10 mins or below, it was used for detection of the analytes.

For electrochemical regeneration, a two electrode setup was used and the voltage of +0.3 V (10 minute) was applied between the gold sensor chip as the working electrode and the inbuilt CE electrode of the QCM electrochemical cell. During this process, the 50 mM MES buffer pH 7 was allowed to flow continuously for 10 min to wash away the detached analytes from the surface of sensor. During the entire process, the temperature of the QCM chamber was adjusted to desired temperature (25° C. or 37° C.) with the inbuilt temperature controller.

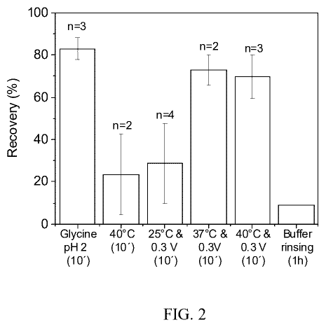

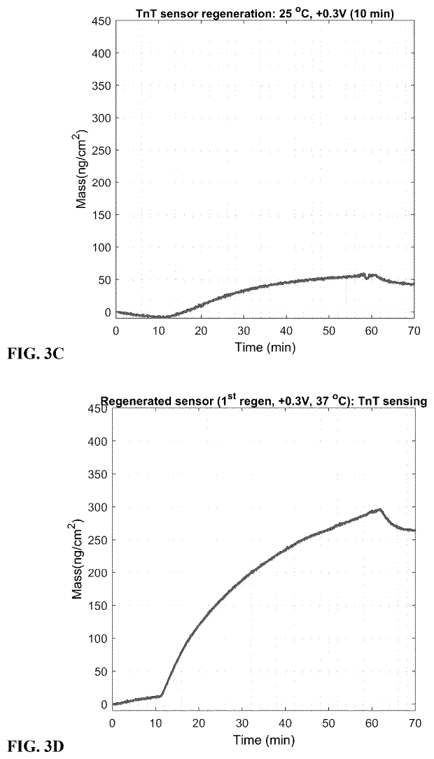

Already a short incubation time of the immunosensor of 10 min comprising antibodies being covalently attached to an electroconductive surface under a potential applied of +300 mV 1 (being the working electrode comprising the electroconductive surface to which the Fab antibody fragments, anti-TSH F(ab′)2 or anti-TnT Fab antibody fragments are coupled via EDC-NHS chemistry as indicated above) and a temperature over 25° C. leads to a regeneration of the mentioned immunosensor (with recoveries over 60% for tested temperatures, see examples 2 and, FIG. 2 and FIG. 4).

### Example 2: TSH-Immunosensor Regeneration

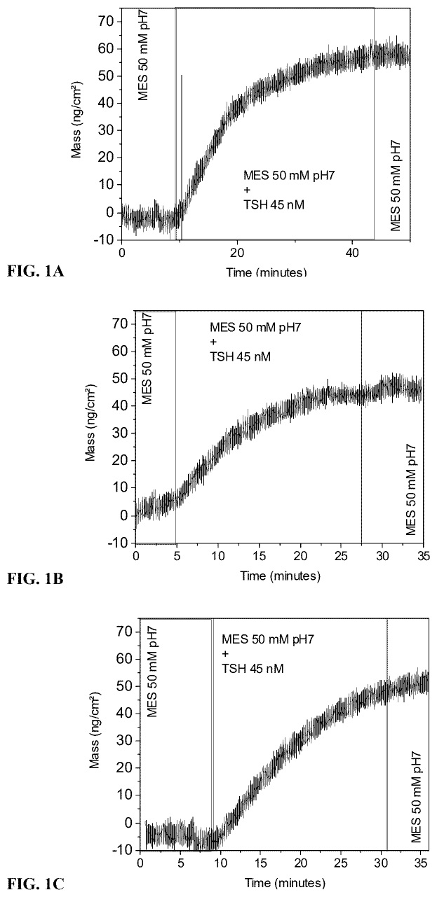

45 nM TSH was used for binding to a new sensor and to regenerated sensors. Using a new, i.e. a fresh sensor 57 ng of TSH was bound by the sensor as determined by electrochemical QCM (e-QCM, see FIG. 1A). The sensor was regenerated using either an electrochemically regeneration protocol or conventional glycine regeneration. The electrochemical regeneration of the TSH sensor was performed at 40° C. and applying 0.3 V for 10 minutes following the regeneration protocol as described herein above in more detail. After regeneration, 46 ng of TSH could be bound to the regenerated sensor representing a regenerated binding capacity of 80.7% compared to a new sensor (FIG. 1B). As a comparative example, a TSH sensor was regenerated using a conventional glycine regeneration protocol as described herein above in more detail. Briefly, regeneration was performed using a glycine treatment at pH 2 for 10 minutes. Thereafter, the regenerated sensor could bind 51 ng of TSH representing a regenerated binding capacity of 89.5% compared to a new sensor (FIG. 1C).

The regenerated binding capacity using different regeneration approaches has been assessed (FIG. 2). A potential of +0.3 V has been applied combined with a temperature of 25° C., 37° C. or 40° C. As an additional experiment, a temperature of 40° C. has been applied without application of a potential. The percentage of sensor recovery was calculated as the ratio of the amount of analyte (TSH) bound to the immunosensor after regeneration to the amount of the analyte bound by a fresh immunosensor multiplied by 100%. Our results show that higher temperatures lead to increased TSH sensor regeneration.

### Example 3: Cardiac Troponin T-immunosensor Regeneration

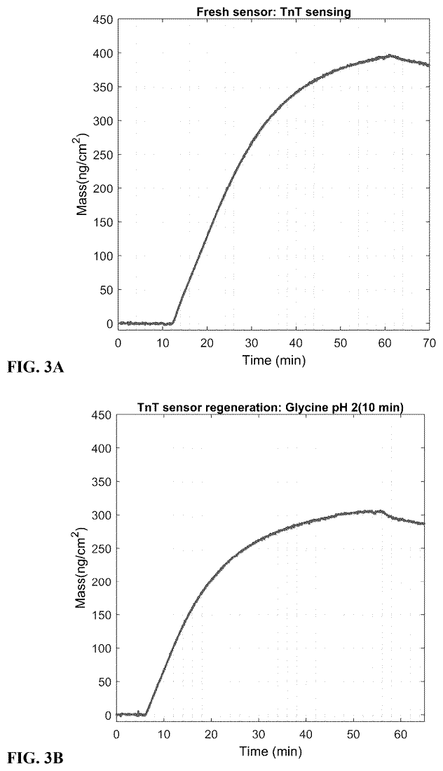

Regeneration of a sensor for cardiac Troponin T (TnT). 44 nM TnT was used for binding to a new sensor and to regenerated sensors. Using a new, i.e. a fresh sensor 394 ng of TnT was bound by the sensor as determined by e-QCM (see FIG. 3A). The sensor was regenerated using either an electrochemically regeneration protocol or conventional glycine regeneration. As a comparative example, the TnT sensor was regenerated using a conventional glycine regeneration protocol as described herein above in more detail. Briefly, regeneration was performed using a glycine treatment at pH 2 for 10 minutes. Thereafter, the regenerated sensor could bind 304.4 ng of TnT representing a regenerated binding capacity of 77.25% compared to a new sensor (FIG. 3B). The electrochemical regeneration of the TnT sensor was performed at 25° C. and applying 0.3 V for 10 minutes following the regeneration protocol as described herein above in more detail. After regeneration, 63.26 ng of TnT could be bound to the regenerated sensor representing a regenerated binding capacity of 16.05% compared to the binding capacity of a new (fresh) sensor (FIG. 3C). The temperature for the electrochemical regeneration was set to 37° C. (FIG. 3D), then 285.94 ng of TnT could be bound to the regenerated sensor. The regenerated binding capacity increased significantly to 76.34%.

A comparison of the different regeneration approaches for TnT sensors is depicted in FIG. 4. The percentage of sensor recovery is calculated as the ratio of the amount of analyte (TSH) bound to the immunosensor after regeneration to the amount of the analyte bound by a fresh immunosensor multiplied by 100%. Our results show that as for TSH sensor regeneration (example 2) higher temperatures lead to a significant increase sensor regeneration for TnT sensors.

### Example 4: Multiple Regeneration Cycles

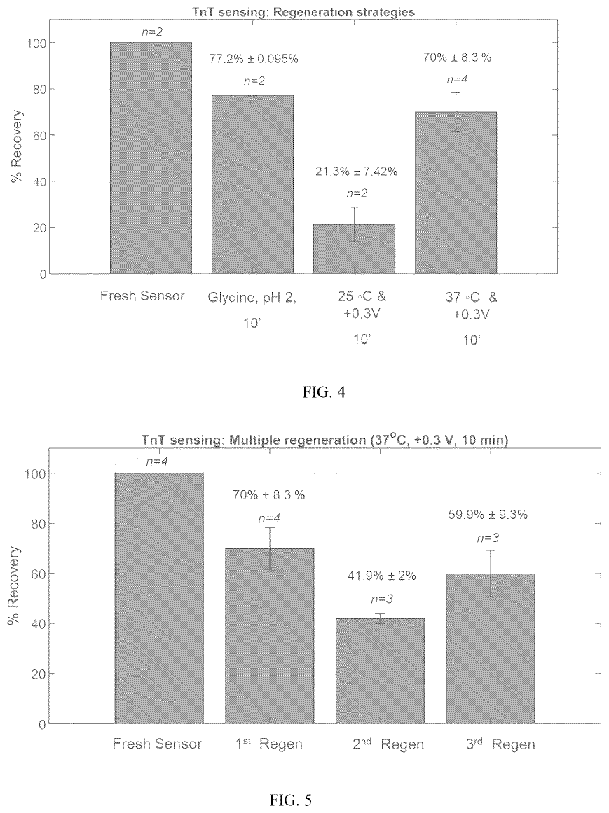

A TnT sensor was electrochemically regenerated as in example 3, but for multiple times, i.e up to three times. For second and third regenerations, the regeneration procedure as outlined above was repeated two and three times respectively. The results are shown in FIG. 5. The results show that using our regeneration approach, even multiple regeneration cycles of the same sensor are possible.

### LITERATURE CITED

U.S. Pat. No. 7,118,922 B1
Asanov et al., 1998, Anal. Chem. 70, 1156-1163)
Brod et al., 2008 Sensors and Actuators B, 128, 560-565
Khoo, et al., 2016, Sensors and Actuators B, 224, 683-691
The Immunoassay Handbook, Fourth Edition, Elsevier 2013, ISBN 978-0-08-097037-0, chapter 2.12

1. A method for regenerating an immunosensor comprising at least one polypeptide attached to an electroconductive surface of said immunosensor, wherein the at least one polypeptide is capable of specifically binding an analyte to be detected by the immunosensor, said method comprising the step of applying to the immunosensor at a temperature selected from the temperature range from about 35° C. to about 42° C. a positive electrical potential of about 0.3 V on said electroconductive surface of the immunosensor for a time sufficient to allow regeneration.

2. The method of claim 1, wherein said at least one polypeptide has bound said analyte prior to carrying out the method.

3. The method of claim 1, wherein said polypeptide which is capable of specifically binding said analyte to be detected by the immunosensor is selected from the group consisting of: an antibody, a receptor protein, a matrix protein, an enzyme, and a nucleic acid binding protein.

4. The method of claim 1, wherein said time is selected from the range of about 8 to about 12 min.

5. The method of claim 4, wherein said time is about 10 min.

6. The method of claim 1, wherein the binding capacity of the immunosensor can be recovered up to at least 60%, at least 70% or at least 75% compared to a control immunosensor.

7. The method of claim 1, wherein said at least one polypeptide is specifically recognizing a cardiac troponin and said analyte is said cardiac troponin.

8. The method of claim 7, wherein said cardiac troponin is troponin T.

9. The method of claim 1, wherein said method comprises at least once a further step of measuring using the regenerated immunosensor and a further step of applying to the immunosensor at a temperature selected from the temperature range from about 35° C. to about 42° C. a positive electrical potential of about 0.3 V on said electro conductive surface of the immunosensor for a time sufficient to allow regeneration after measuring.

10. A system adapted to perform the method according to claim 1 comprising:
i) a device comprising:
at least one immunosensor comprising at least one polypeptide attached to an electroconductive surface of said immunosensor, wherein the at least one polypeptide is capable of specifically binding an analyte to be detected by the immunosensor, and
at least one control element capable of applying a positive electrical potential of about 0.3 V on said electroconductive surface of the immunosensor for a time sufficient to allow regeneration;
ii) at least one electronic control element capable of controlling at least the parameters applied in the method.

11. The system of claim 10, wherein the at least one electronic control element is further capable controlling the temperature.

12. The system of claim 10, wherein said polypeptide which is capable of specifically binding said analyte to be detected by the immunosensor is selected from the group consisting of: an antibody, a receptor protein, a matrix protein, an enzyme, and a nucleic acid binding protein.

13. The system of claim 10, wherein said immunosensor is a regenerated immunosensor obtained by the a method for generating an immunosensor comprising an electroconductive surface comprising applying to the electroconductive surface.

14. The system of claim 10, wherein said at least one electronic control element is a microcontroller capable of controlling at least the positive electrical potential and the time of applying the potential.

15. A method for generating an immunosensor comprising an electroconductive surface comprising applying to the electroconductive surface a temperature selected from the temperature range of from about 35° C. to about 42° C. and a positive electrical potential of about 0.3 V.
